(19) United States
(12) Patent Application Publication
(10) Pub. No.: US 2025/0391535 A1
(43) Pub. Date: Dec. 25, 2025

(54) AUTOMATED SINGLE-DOSE DISPENSING OF OPIOID ADDICTION MEDICATION

(71) Applicant: OPIO CONNECT, INC., Kalispell, MT (US)

(72) Inventors: Amber Norbeck, Kalispell, MT (US); Michael Pokorny, Kalispell, MT (US); Thomas Hoffmann, Kalispell, MT (US)

(21) Appl. No.: 19/211,759

(22) Filed: May 19, 2025

Related U.S. Application Data
(60) Provisional application No. 63/649,075, filed on May 17, 2024.

(51) Int. Cl. G16H 20/13; A61J 1/00; A61J 7/00

(52) U.S. Cl. CPC G16H 20/13; A61J 1/00; A61J 7/0076

(57) ABSTRACT

A system for dispensing prepackaged single doses of a medication. The system includes a carousel made up of columns each containing single doses sharing a dosage size and a medication type, configured to rotate about an axis. A pushing mechanism is coupled to the columns, configured to push a selected single dose from a column onto the verification platform. The system further includes a biometric authentication mechanism for determining an identity of a patient based on biometric data to find a dosage order corresponding to their identity, and directing the rotation mechanism to rotate the dose in the dosage order in-line with the pushing mechanism. The system further includes a mechanism configured to dispense the selected single dose if the selected single dose matches the dosage order, and push the selected single dose into a rejection reservoir if the selected single dose does not match the dosage order.

### CROSS-REFERENCE TO RELATED APPLICATIONS

This application is a non-provisional and claims benefit of U.S. Provisional Application No. 63/649,075 filed May 17, 2024, the specification of which is incorporated herein in its entirety by reference.

### FIELD OF THE INVENTION

The present invention is directed to securely delivering prepackaged single doses of opioid addiction medication to users in correctional facilities.

### BACKGROUND OF THE INVENTION

In correctional facilities, a nurse is required to be on staff to pour out and deliver medication for opioid addiction to incarcerated patients. This is a major bottleneck and may result in some patients not receiving their medication on time or at all. This is an even bigger issue in more remote areas where it is more difficult to get nurses certified for opioid addiction treatment on staff. Thus, there exists a present need for a system that allows for the secure delivery of prepackaged single doses of opioid addiction medication to patients in correctional facilities.

### BRIEF SUMMARY OF THE INVENTION

It is an objective of the present invention to provide systems and methods that allow for secure delivery of prepackaged single doses of opioid addiction medication to users in correctional facilities, as specified in the independent claims. Embodiments of the invention are given in the dependent claims. Embodiments of the present invention can be freely combined with each other if they are not mutually exclusive.

The present invention features a system for dispensing prepackaged single doses of a medication. The system may comprise a housing and a verification platform disposed within the housing. The system may further comprise a carousel disposed within the housing, comprising a plurality of columns, each one configured to contain one or more single doses sharing a dosage size and a medication type. The carousel may further comprise a cavity defined by the plurality of columns, a rotation mechanism configured to rotate the plurality of columns about an axis defined by the cavity, and a pushing mechanism disposed within the cavity, configured to push a selected single dose from a column onto the verification platform.

The system may further comprise a biometric authentication mechanism, configured to accept biometric data from a patient, verify their identity, identify a dosage order corresponding to their identity, and direct the rotation mechanism to rotate the plurality of columns such that the selected single dose corresponding to the dosage order aligns with the pushing mechanism. The system may further comprise a verification mechanism configured to verify that the selected single dose matches the dosage order. The system may further comprise an ejection mechanism, configured to dispense the selected single dose if the verification mechanism determines that the selected single dose matches the dosage order. The system may further comprise a rejection mechanism operatively coupled to the verification platform and the verification mechanism, configured to push the selected single dose into a rejection reservoir if the verification mechanism determines that the selected single dose does not match the dosage order.

The present invention features a method for dispensing prepackaged single doses of a medication. The method may comprise receiving, by a biometric authentication mechanism, biometric data from a patient, and verifying, by the biometric authentication mechanism, an identity of the patient based on the biometric data. The method may further comprise identifying, by the biometric authentication mechanism, a dosage order corresponding to the identity of the patient, the dosage order corresponding to a selected single dose. The method may further comprise rotating, by a carousel, a plurality of columns each configured to contain one or more single doses sharing a dosage size and a medication type, such that the selected single dose corresponding to the dosage order aligns with a pushing mechanism.

The method may further comprise pushing, by the pushing mechanism, the selected single dose onto a verification platform and verifying, by a verification mechanism, that the selected single dose matches the dosage order. The method may further comprise dispensing, by an ejection mechanism, the selected single dose if the verification mechanism determines that the selected single dose matches the dosage order. The method may further comprise pushing, by a rejection mechanism, the selected single dose into a rejection reservoir if the verification mechanism determines that the selected single dose does not match the dosage order.

One of the unique and inventive technical features of the present invention is the implementation of an automated verification and rejection system in a machine for autonomously dispensing medication. Without wishing to limit the invention to any theory or mechanism, it is believed that the technical feature of the present invention advantageously provides for an efficient and fully automated process for delivering prepackaged single doses of opioid addiction medication to patients with minimal human intervention required. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a view of the system for dispensing prepackaged single doses of a medication of the present invention.

FIG. 1B shows a view of the system for dispensing prepackaged single doses of a medication in use.

FIG. 2 shows a flow chart of a method for dispensing prepackaged single doses of a medication of the present invention.

FIG. 3 shows a view of the system for dispensing prepackaged single doses of a medication in an embodiment where the system is disposed in a safe.

FIG. 4 shows a view of a dose being dispensed by the system of the present invention.

FIG. 5 shows a view of a dose being rejected by the system of the present invention.

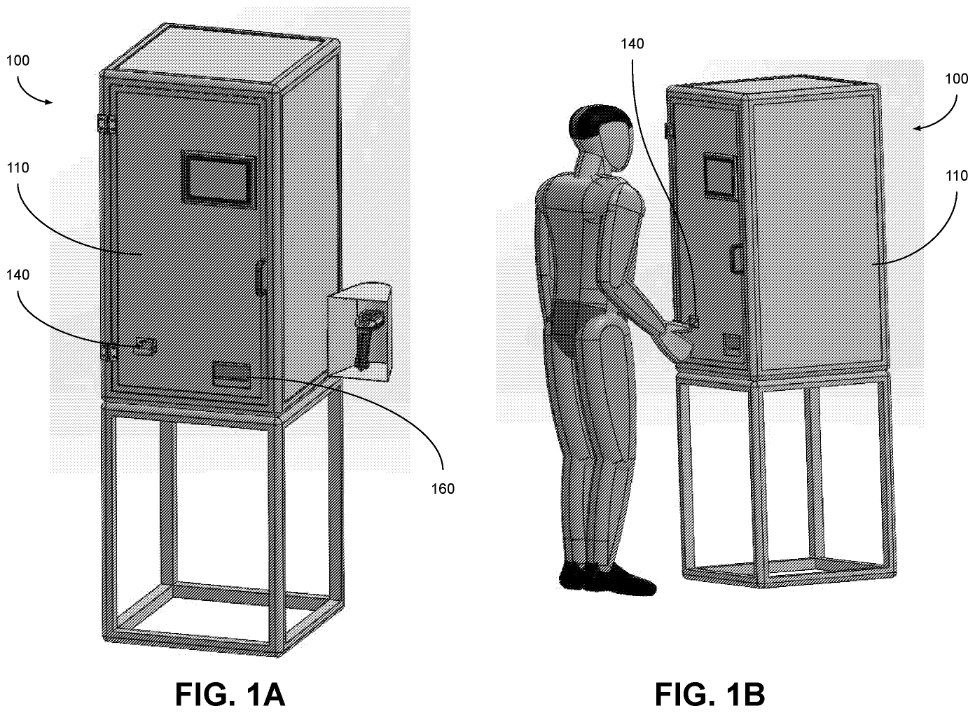

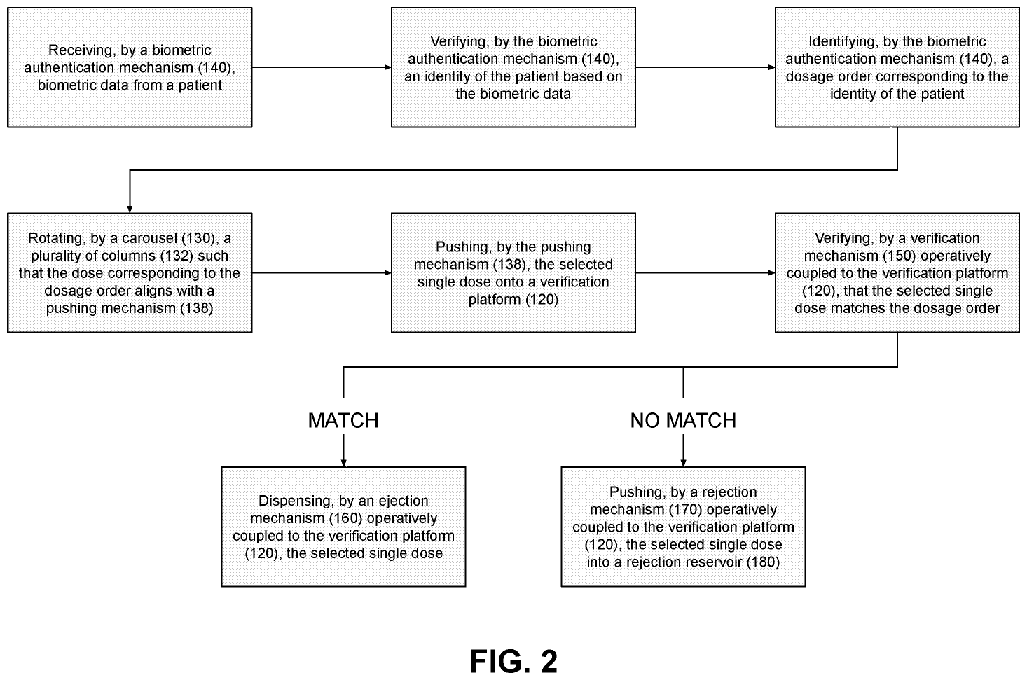

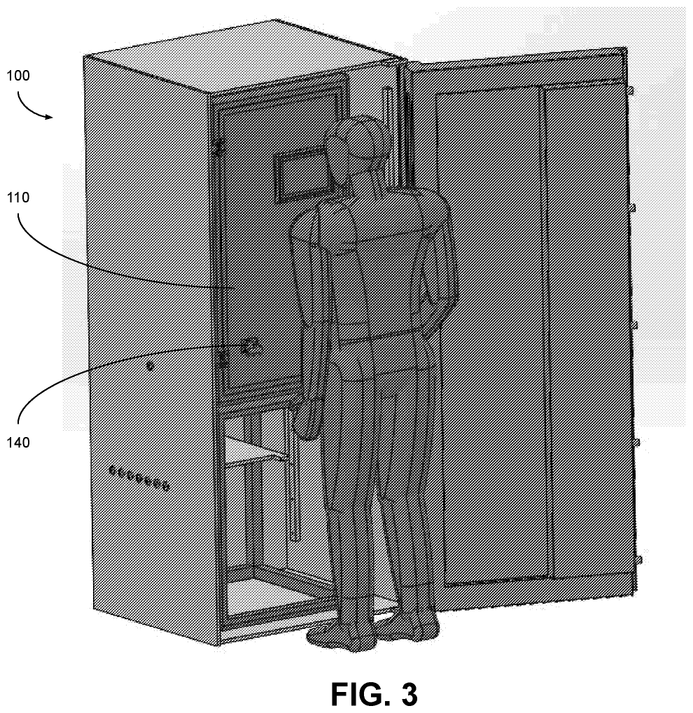

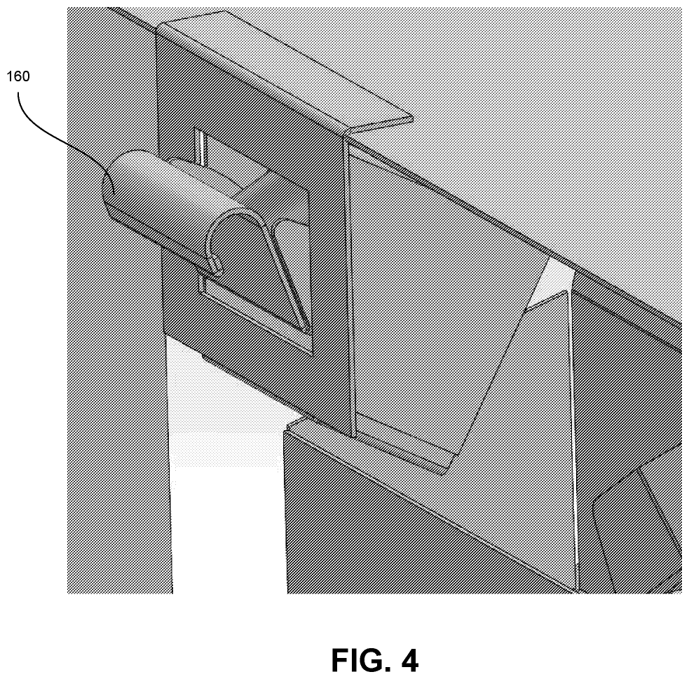

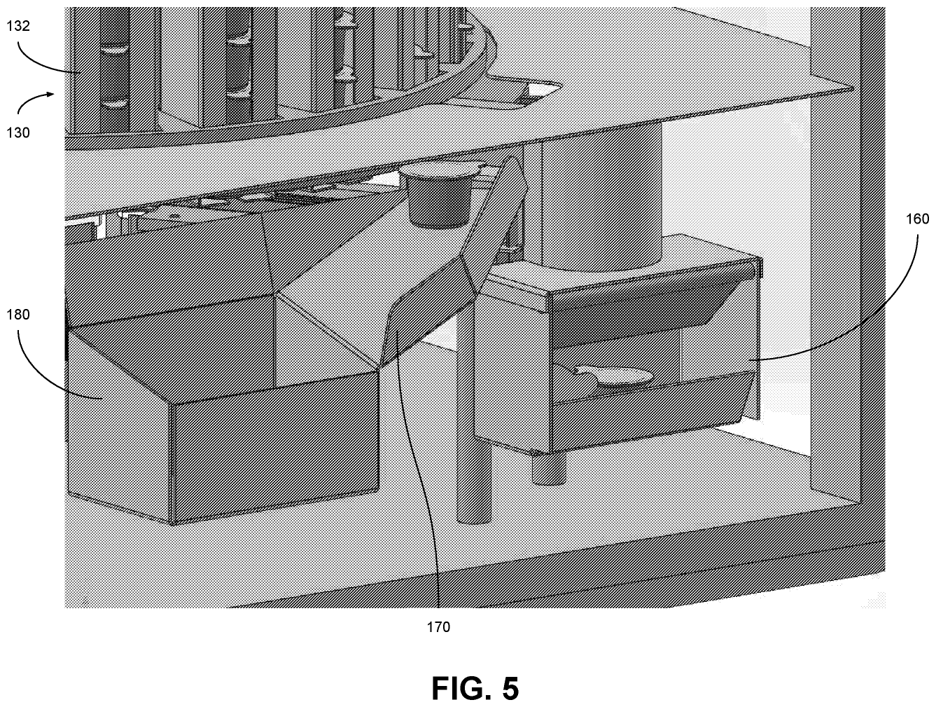

### DETAILED DESCRIPTION OF THE INVENTION

Following is a list of elements corresponding to a particular element referred to herein:
100 system
110 housing
120 verification platform
130 carousel
132 columns
134 cavity
136 rotation mechanism
138 pushing mechanism
140 biometric authentication component
150 verification mechanism
160 ejection mechanism
165 automated arm component
170 rejection mechanism
180 rejection reservoir

The present invention features a system (100) for dispensing prepackaged single doses of a medication. In some embodiments, the system (100) may comprise a housing (110). The system (100) may further comprise a verification platform (120) disposed within the housing (110). The system (100) may further comprise a carousel (130) disposed within the housing (110). In some embodiments, the carousel (130) may comprise a plurality of columns (132), each column configured to contain one or more single doses. The one or more single doses of each column may share a dosage size and a medication type. The carousel (130) may further comprise a cavity (134) defined by the plurality of columns (132). The carousel (130) may further comprise a rotation mechanism (136) configured to rotate the plurality of columns (132) about an axis defined by the cavity (134). The carousel (130) may further comprise a pushing mechanism (138) disposed within the cavity (134), configured to push a selected single dose from a column onto the verification platform (120).

In some embodiments, the system (100) may further comprise a biometric authentication mechanism (140) communicatively coupled to the carousel (130), configured to accept biometric data from a patient, verify an identity of the patient, identify a dosage order corresponding to the identity of the patient, wherein the dosage order corresponds to the selected single dose, and direct the rotation mechanism (136) to rotate the plurality of columns (132) such that the selected single dose corresponding to the dosage order aligns with the pushing mechanism (138). The system (100) may further comprise a verification mechanism (150) operatively coupled to the verification platform (120), configured to verify that the selected single dose matches the dosage order. The system (100) may further comprise an ejection mechanism (160) operatively coupled to the verification platform (120) and the verification mechanism (150), configured to dispense the selected single dose if the verification mechanism (150) determines that the selected single dose matches the dosage order. The system (100) may further comprise a rejection mechanism (170) operatively coupled to the verification platform (120) and the verification mechanism (150), configured to push the selected single dose into a rejection reservoir (180) if the verification mechanism (150) determines that the selected single dose does not match the dosage order.

In some embodiments, the biometric authentication mechanism (140) may comprise a fingerprint scanner, a retina scanner, a facial recognition system, or a combination thereof. In some embodiments, the medication may comprise methadone, buprenorphine, or a combination thereof. In some embodiments, the verification mechanism (150) may comprise a barcode scanner. In some embodiments, the ejection mechanism (160) may comprise an automated arm component. In some embodiments, the rejection mechanism (170) may comprise an automated arm component. In some embodiments, the pushing mechanism (138) may comprise a motorized device comprising a motor, a slot, and a rod operatively coupled to the motor, disposed in the slot, wherein the motor is configured to extend the rod from the slot and retract the rod into the slot when actuated. In some embodiments, the rotation mechanism may comprise a motorized platform comprising a platform and a motor operatively coupled to the platform, configured to rotate the platform when actuated. In some embodiments, the system (100) of the present invention may be disposed in a secure safe.

The present invention features a method for dispensing prepackaged single doses of a medication. In some embodiments, the method may comprise receiving, by a biometric authentication mechanism (140), biometric data from a patient. The method may further comprise verifying, by the biometric authentication mechanism (140), an identity of the patient based on the biometric data. The method may further comprise identifying, by the biometric authentication mechanism (140), a dosage order corresponding to the identity of the patient. The dosage order may correspond to a selected single dose. The method may further comprise rotating, by a carousel (130) communicatively coupled to the biometric authentication mechanism (140), a plurality of columns (132), each column configured to contain one or more single doses, such that the selected single dose corresponding to the dosage order aligns with a pushing mechanism (138) operatively coupled to the plurality of columns (132). The one or more single doses of each column may share a dosage size and a medication type.

The method may further comprise pushing, by the pushing mechanism (138), the selected single dose onto a verification platform (120). The method may further comprise verifying, by a verification mechanism (150) operatively coupled to the verification platform (120), that the selected single dose matches the dosage order. The method may further comprise dispensing, by an ejection mechanism (160) operatively coupled to the verification platform (120), the selected single dose if the verification mechanism (150) determines that the selected single dose matches the dosage order. The method may further comprise pushing, by a rejection mechanism (170) operatively coupled to the verification platform (120), the selected single dose into a rejection reservoir (180) if the verification mechanism (150) determines that the selected single dose does not match the dosage order.

In some embodiments, the biometric authentication mechanism (140) may comprise a fingerprint scanner, a retina scanner, a facial recognition system, or a combination thereof. In some embodiments, the medication may comprise methadone, buprenorphine, or a combination thereof. In some embodiments, the verification mechanism (150) may comprise a barcode scanner. In some embodiments, the ejection mechanism (160) may comprise an automated arm component. In some embodiments, the rejection mechanism (170) may comprise an automated arm component. In some embodiments, the pushing mechanism (138) may comprise a motorized device comprising a motor, a slot, and a rod operatively coupled to the motor, disposed in the slot, wherein the motor is configured to extend the rod from the slot and retract the rod into the slot when actuated. In some embodiments, the rotation mechanism may comprise a motorized platform comprising a platform and a motor operatively coupled to the platform, configured to rotate the platform when actuated.

The present invention features a system (100) for dispensing prepackaged single doses of a medication. In some embodiments, the system (100) may comprise a housing (110). The system (100) may further comprise a verification platform (120) disposed within the housing (110). The system (100) may further comprise a carousel (130) disposed within the housing (110). In some embodiments, the carousel (130) may comprise a plurality of columns (132), each column configured to contain one or more single doses. The one or more single doses of each column may share a dosage size and a medication type. The carousel (130) may further comprise a cavity (134) defined by the plurality of columns (132). The carousel (130) may further comprise a rotation mechanism (136) configured to rotate the plurality of columns (132) about an axis defined by the cavity (134). The carousel (130) may further comprise a pushing mechanism (138) disposed within the cavity (134), configured to push a selected single dose from a column onto the verification platform (120), the selected single dose comprising a barcode unique to the selected single dose.

In some embodiments, the system (100) may further comprise a computing device (1000) communicatively coupled to the carousel (130). The computing device (1000) may comprise a biometric authentication component (140) configured to accept biometric data from a patient. The computing device (1000) may further comprise a processor (1002) operatively coupled to the biometric authentication component (140), configured to execute computer-readable instructions. The computing device (1000) may further comprise a memory component (1004) operatively coupled to the processor (1002), comprising computer-readable instructions. The computer-readable instructions may comprise accepting the biometric data from the biometric authentication component (140), verifying an identity of the patient based on the biometric data, identifying a dosage order corresponding to the identity of the patient, wherein the dosage order corresponds to the selected single dose, and directing the rotation mechanism (136) to rotate the plurality of columns (132) such that the selected single dose corresponding to the dosage order aligns with the pushing mechanism (138).

The system (100) may further comprise a barcode scanner (150) operatively coupled to the verification platform (120), configured to scan the barcode of the selected single dose and verify that the selected single dose matches the dosage order. The system (100) may further comprise an automated arm component (165) operatively coupled to the verification platform (120) and the barcode scanner (150), configured to dispense the selected single dose if the barcode scanner (150) determines that the selected single dose matches the dosage order, and push the selected single dose into a rejection reservoir (180) if the barcode scanner (150) determines that the selected single dose does not match the dosage order.

In some embodiments, the biometric authentication component (140) may comprise a fingerprint scanner, a retina scanner, a facial recognition system, or a combination thereof. In some embodiments, the medication may comprise methadone, buprenorphine, or a combination thereof. In some embodiments, the memory component (1004) may further comprise computer-readable instructions for operating the automated arm component (165) to dispense the selected single dose and push the selected single dose into a rejection reservoir (180). In some embodiments, the pushing mechanism (138) may comprise a motorized device comprising a motor, a slot, and a rod operatively coupled to the motor, disposed in the slot, wherein the motor is configured to extend the rod from the slot and retract the rod into the slot when actuated. In some embodiments, the rotation mechanism may comprise a motorized platform comprising a platform and a motor operatively coupled to the platform, configured to rotate the platform when actuated. In some embodiments, the system (100) of the present invention may be disposed in a secure safe.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A system (100) for dispensing prepackaged single doses of a medication, the system (100) comprising:
a. a housing (110);
b. a verification platform (120) disposed within the housing (110);
c. a carousel (130) disposed within the housing (110), the carousel (130) comprising:
i. a plurality of columns (132), each column configured to contain one or more single doses;
wherein the one or more single doses of each column share a dosage size and a medication type;
ii. a cavity (134) defined by the plurality of columns (132);
iii. a rotation mechanism (136) configured to rotate the plurality of columns (132) about an axis defined by the cavity (134); and
iv. a pushing mechanism (138) disposed within the cavity (134), configured to push a selected single dose from a column onto the verification platform (120);
d. a biometric authentication mechanism (140) communicatively coupled to the carousel (130), configured to:
i. accept biometric data from a patient;
ii. verify an identity of the patient;
iii. identify a dosage order corresponding to the identity of the patient, wherein the dosage order corresponds to the selected single dose; and
iv. direct the rotation mechanism (136) to rotate the plurality of columns (132) such that the selected single dose corresponding to the dosage order aligns with the pushing mechanism (138);
e. a verification mechanism (150) operatively coupled to the verification platform (120), configured to verify that the selected single dose matches the dosage order;
f. an ejection mechanism (160) operatively coupled to the verification platform (120) and the verification mechanism (150), configured to dispense the selected single dose if the verification mechanism (150) determines that the selected single dose matches the dosage order; and
g. a rejection mechanism (170) operatively coupled to the verification platform (120) and the verification mechanism (150), configured to push the selected single dose into a rejection reservoir (180) if the verification mechanism (150) determines that the selected single dose does not match the dosage order.

2. The system (100) of claim 1, wherein the biometric authentication mechanism (140) comprises a fingerprint scanner, a retina scanner, a facial recognition system, or a combination thereof.

3. The system (100) of claim 1, wherein the medication comprises methadone, buprenorphine, or a combination thereof.

4. The system (100) of claim 1, wherein the verification mechanism (150) comprises a barcode scanner.

5. The system (100) of claim 1, wherein the ejection mechanism (160) comprises an automated arm component.

6. The system (100) of claim 1, wherein the rejection mechanism (170) comprises an automated arm component.

7. The system (100) of claim 1, wherein the pushing mechanism (138) comprises a motorized device comprising a motor, a slot, and a rod operatively coupled to the motor, disposed in the slot, wherein the motor is configured to extend the rod from the slot and retract the rod into the slot when actuated.

8. A method for dispensing prepackaged single doses of a medication, the method comprising:
a. receiving, by a biometric authentication mechanism (140), biometric data from a patient;
b. verifying, by the biometric authentication mechanism (140), an identity of the patient based on the biometric data;
c. identifying, by the biometric authentication mechanism (140), a dosage order corresponding to the identity of the patient, wherein the dosage order corresponds to a selected single dose;
d. rotating, by a carousel (130) communicatively coupled to the biometric authentication mechanism (140), a plurality of columns (132), each column configured to contain one or more single doses, such that the selected single dose corresponding to the dosage order aligns with a pushing mechanism (138) operatively coupled to the plurality of columns (132), wherein the one or more single doses of each column share a dosage size and a medication type;
e. pushing, by the pushing mechanism (138), the selected single dose onto a verification platform (120);
f. verifying, by a verification mechanism (150) operatively coupled to the verification platform (120), that the selected single dose matches the dosage order;
g. dispensing, by an ejection mechanism (160) operatively coupled to the verification platform (120), the selected single dose if the verification mechanism (150) determines that the selected single dose matches the dosage order; and
h. pushing, by a rejection mechanism (170) operatively coupled to the verification platform (120), the selected single dose into a rejection reservoir (180) if the verification mechanism (150) determines that the selected single dose does not match the dosage order.

9. The method of claim 8, wherein the biometric authentication mechanism (140) comprises a fingerprint scanner, a retina scanner, a facial recognition system, or a combination thereof.

10. The method of claim 8, wherein the medication comprises methadone, buprenorphine, or a combination thereof.

11. The method of claim 8, wherein the verification mechanism (150) comprises a barcode scanner.

12. The method of claim 8, wherein the ejection mechanism (160) comprises an automated arm component.

13. The method of claim 8, wherein the rejection mechanism (170) comprises an automated arm component.

14. The method of claim 8, wherein the pushing mechanism (138) comprises a motorized device comprising a motor, a slot, and a rod operatively coupled to the motor, disposed in the slot, wherein the motor is configured to extend the rod from the slot and retract the rod into the slot when actuated.

15. A system (100) for dispensing prepackaged single doses of a medication, the system (100) comprising:
a. a housing (110);
b. a verification platform (120) disposed within the housing (110);
c. a carousel (130) disposed within the housing (110), the carousel (130) comprising:
i. a plurality of columns (132), each column configured to contain one or more single doses;
wherein the one or more single doses of each column share a dosage size and a medication type;
ii. a cavity (134) defined by the plurality of columns (132);
iii. a rotation mechanism (136) configured to rotate the plurality of columns (132) about an axis defined by the cavity (134); and
iv. a pushing mechanism (138) disposed within the cavity (134), configured to push a selected single dose from a column onto the verification platform (120), the selected single dose comprising a barcode unique to the selected single dose;
d. a computing device (1000) communicatively coupled to the carousel (130), comprising:
i. a biometric authentication component (140) configured to accept biometric data from a patient;
ii. a processor (1002) operatively coupled to the biometric authentication component (140), configured to execute computer-readable instructions; and
iii. a memory component (1004) operatively coupled to the processor (1002), comprising computer-readable instructions for:
A. accepting the biometric data from the biometric authentication component (140);
B. verifying an identity of the patient based on the biometric data;
C. identifying a dosage order corresponding to the identity of the patient, wherein the dosage order corresponds to the selected single dose; and
D. directing the rotation mechanism (136) to rotate the plurality of columns (132) such that the selected single dose corresponding to the dosage order aligns with the pushing mechanism (138);
e. a barcode scanner (150) operatively coupled to the verification platform (120), configured to scan the barcode of the selected single dose and verify that the selected single dose matches the dosage order;
f. an automated arm component (165) operatively coupled to the verification platform (120) and the barcode scanner (150), configured to dispense the selected single dose if the barcode scanner (150) determines that the selected single dose matches the dosage order, and push the selected single dose into a rejection reservoir (180) if the barcode scanner (150) determines that the selected single dose does not match the dosage order.

16. The system (100) of claim 15, wherein the biometric authentication component (140) comprises a fingerprint scanner, a retina scanner, a facial recognition system, or a combination thereof.

17. The system (100) of claim 15, wherein the medication comprises methadone, buprenorphine, or a combination thereof.

18. The system (100) of claim 15, wherein the memory component (1004) further comprises computer-readable instructions for operating the automated arm component (165) to dispense the selected single dose and push the selected single dose into a rejection reservoir (180).

19. The system (100) of claim 15, wherein the pushing mechanism (138) comprises a motorized device comprising a motor, a slot, and a rod operatively coupled to the motor, disposed in the slot, wherein the motor is configured to extend the rod from the slot and retract the rod into the slot when actuated.
